(19) United States
(12) Patent Application Publication
(10) Pub. No.: US 2010/0291621 A1
(43) Pub. Date: Nov. 18, 2010

(54) ANAEROBIC PROCESS

(75) Inventors: Jeremy Cooper, Cleveland (GB); Christopher J. Dowle, Cleveland (GB); Stephen Donegan, Cleveland (GB); Michael K. Theodorou, Ceredigion (GB); Bernd Van Houten, Ceredigion (GB)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: CPI INNOVATION SERVICES LIMITED

(21) Appl. No.: 12/735,030

(22) PCT Filed: Dec. 11, 2008
(86) PCT No.: PCT/GB2008/004103

§ 371 Date: Jul. 27, 2010

(30) Foreign Application Priority Data
Dec. 11, 2007 (GB) 0724179.7

(51) Int. Cl. C12P 1/00; C12N 9/00; C12M 1/00

(52) U.S. Cl. 435/41; 435/183; 435/289.1

(57) ABSTRACT

An anaerobic process uses a continuous oscillatory flow reactor (1) having a reaction tube with baffles (7) along its length. Fluid material is introduced into the tube through a circulating loop (41) and control valve (28) configuration. The fluid comprises the following components: a finely divided substrate material, e.g. of ground straw, and facultative and/or obligate anaerobic organisms such as rumen organisms. The fluid material passes through the tube at a speed that allows anaerobic fermentation to take place, and is caused to oscillate with respect to the baffles. The microbial biomass, extra-cellular enzymes and liquid and gaseous fermentation end-products can be collected at outlets (5, 9). The oscillating-flow system can be used to grow obligately anaerobic microorganisms and is ideal for fermentation processes because of its thorough mixing at low shear force and controllable conditions. In it rumen processes in particular can be run, resulting in a much faster digestion than in conventional anaerobic digesters.

### BACKGROUND OF THE INVENTION

Fermentation processes are used to break down organic-based feedstock into smaller, more useful molecules via microbial transformations and process purifications. They are generally carried out in batch or fed-batch reactors, and are usually preceded by a period of aerobic microbial growth. They usually employ axenic microorganisms (i.e. a single strain) or a limited mixed microbial community. For a successful process, good aeration is needed during the growth phase and this is achieved by appropriate mixing and aeration of the culture medium plus microorganism. Aeration usually defines reactor design and limits microbial growth in many processes. Once sufficient biomass is achieved, process conditions are altered, usually by excluding air, to permit fermentation and the formation of useful end-products. The methods for production of penicillin and ethanol are typically described as fermentation processes.

Anaerobic digestion, on the other hand, is a process in which microorganisms grow and break down biodegradable material in the complete absence of oxygen. Both the growth of the microbial population and the production of useful end-products are linked and achieved exclusively by anaerobic fermentation. The microorganisms involved are usually present as mixed microbial communities. The substrates used in anaerobic digestion are often particulate and usually recalcitrant, requiring fibrolytic enzymes for their comprehensive degradation. Anaerobic digestion is widely used to treat wastewater sludge and to biodegrade organic wastes. It provides both volume and mass reduction and is extremely effective at reducing the chemical oxygen demand of the influent material. If harnessed correctly, anaerobic digestion can be used to produce biogas (a methane- and carbon-dioxide-rich gas), making the process suitable for energy production and helping to replace fossil fuels. Anaerobic digestion processes are generally carried out on a commercial scale in large tanks, in one-stage or two-stage batch systems, and the process needs between 10 and 100 days in order to reach an acceptable degree of digestion. The major drawback of these systems is their large size relative to biogas yield (large footprint) and their limited reliability, with impairment of the digestion process often caused by lack of effective digestion and clogging due to the recalcitrant nature of the substrate.

The digestion of plant biomass by mammalian and insect herbivores is also an obligately anaerobic process involving mixed microbial communities living in anoxic ecosystems. These micro-organisms behave rather like the populations in an industrial anaerobic digester, in that growth and end-product formation are linked and fibrolytic enzymes are required for substrate degradation. One other similarity relates to the fact that digestive tract communities are capable of inter-species hydrogen transfer, involving the Archaea, and they also produce methane. The economic importance of ruminant livestock makes the microbial community inhabiting the reticulo-rumen probably the best studied of all mixed-population microbial communities. It is recognised that the ecosystem contains many interesting micro-organisms including the highly fibrolytic anaerobic fungi and bacteria producing volatile fatty acids (VFA), succinate and lactate; currently there is much interest in the ability to harness some of these micro-organisms in industrial-scale processes. Some typical rumen microorganisms include the highly fibrolytic anaerobic fungi, Neocallimastix frontalis and Cycllamyces aberensis and obligately anaerobic bacterial species such as fibrolytic Ruminococcus flavefaciens (fibrolytic), Bacterodes succinogenes (succinate-producing) Butyrivibrio fibrisolvens (hemicellulolytic and butyrate-producing). Hydrogen-utilising, methanogenic Archaea, such as Methanobacterium ruminantium, are also commonplace in the rumen.

Although there are process similarities between anaerobic digestion and the rumen ecosystem, one of the major differences is the rate and extent of fermentation that can be achieved in the rumen. This sets the rumen apart as a fermentation vessel par excellence, demonstrating log-scale improvement over anaerobic digesters by achieving in 1-2 days what an industrial plant can achieve in 10-100 days. The rumen provides an anaerobic environment, constant temperature and pH and good mixing. Unlike the anaerobic digestion batch process, it is an open, continuous ecosystem and well-masticated substrates are delivered at regular intervals in well-buffered saliva and drinking water through the oesophagus. Fermentation end-products are either absorbed to the bloodstream across the rumen wall or flow out for further processing and adsorption down-stream. Thus, by efficient conversion of plant particulate matter, rumen fermentation results in the production of microbial protein and the fermentation end-products, volatile fatty acids (acetate, propionate, butyrate), carbon dioxide and methane. While microbial protein and volatile fatty acids may be considered as platform chemicals, satisfying much of the protein and energy requirements for meat and milk production, the gaseous emissions contribute negatively to carbon footprint.

The rationale behind the superior behaviour of the rumen ecosystem relative to an anaerobic digestion plant is multifarious and complex. Perhaps it is due in part to the presence of certain groups of micro-organisms in the rumen that are absent in an anaerobic digestion plant. The anaerobic fungi in the rumen for example are the most fibrolytic organisms known to man but, because of their obligately anaerobic nature and need to be cultured in an open ecosystem, they are absent in industrial-scale processes. The ability to support the obligately anaerobic microbial community of the rumen as a resilient and responsive population in a reactor with the potential to be exploited in commerce is the main focus of this invention. Mixing at low shear force has been seen to be an important requirement. One system that provides very good mixing without excessive agitation is the oscillating-flow baffle system. The use of mixing equipment such as oscillatory flow tubes with baffles, mixing the liquid as it passes through the reactor, is well known (see for instance WO 2006/136850 by Nitech Solutions Ltd). However, these are generally applied to straightforward chemical reactions, such as hydrogenation, where mixing in of a gas phase is crucial to the reaction being carried out successfully. Although the patent mentioned above does speculate on the possible use in fermentations, no further benefit is claimed, nor data presented nor particular adaptations described.

The inventors have recognised that the oscillatory scheme, when modified to include the injection and ejection of substrates and anaerobic media preparation and operational techniques to ensure that it can maintain anoxic conditions, is suitable for anaerobic digestion and anaerobic microbiological processes, particularly those using obligately anaerobic microbial communities originating from the gut of herbivores, and can be made comparable to the rapid process taking place in the ruminant itself. These communities in ruminant herbivores for example typically contain a variety of obligately anaerobic bacteria, protozoa and fungi that require exacting culture conditions and are killed on exposure to oxygen in air.

According to one aspect of the invention there is provided a method for producing an anaerobic microbiological process, comprising the steps of: providing an oscillatory flow reactor having a reaction tube arranged vertically or horizontally depending on the microbiological process with baffles along its length; introducing fluid material into the tube, the fluid comprising the following components: finely divided (≦2 mm²) particulate substrate/substratum materials; an anaerobic community of micro-organisms, and feed material for the organisms, the feed possibly being identical to the substrate; passing the fluid material through the tube at a speed that allows the anaerobic process to take place, the fluid oscillating with respect to the baffles; and collecting the grown microbial cells, extra-cellular enzymes and gaseous and or liquid fermentation end-products.

According to another aspect of the invention there is provided a preferably continuous oscillatory flow reactor, comprising: a tube, with feed in at one end and discharge at the other, and a baffle-plate system arranged along its length for mixing the contents of the tube by oscillation of the contents with respect to the plates; further including at the feed end of the tube a supply device adapted to feed a suspension of soluble and/or insoluble substrate particles, and a means for introducing anaerobic organisms, under anaerobic conditions. Alternatively or additionally the substrate can include surfaces of the baffles themselves.

The tube can be arranged vertically, horizontally or at an intermediate inclination, depending on the microbiological process. There may be an aerobic run before the anaerobic conditions set in.

In addition to the oscillatory flow tubes with baffles for mixing and keeping particles in suspension, preferably two further features are preferably present to make the apparatus and method suitable for growth of obligately anaerobic microorganisms. These are the substrate-recycling process, where the particulate material is circulated to maintain suspension until it is ready for injection into the reactor, and the synchronous operation of valves for adding new feed and removing spent liquid. Collectively, these features prevent particles from settling out in feed-lines prior to reaching the reactor and to permit them to be injected to and ejected from the reactor in precise quantities and at precise times. The synchronous valve system advantageously prevents ingress of oxygen (in air) to the reactor, which would otherwise be detrimental to anaerobic microbial processes in the anoxic reactor environment.

The baffles themselves can be smooth or they can themselves have a finely porous or divided surface and act as a substrate for the organisms. Many of the obligate or facultative micro-organisms, whether genetically modified or not, need a substrate on which to grow. The substrate can be the particulate material itself or, alternatively or additionally, the baffles as mentioned. Additional feed can also be held in suspension or dissolved.

The apparatus allows the addition of material either at the inlet end, for instance at the bottom of the tube, or at any intermediate point in the tube. Microbial biomass, extra-cellular enzymes and liquid and gaseous fermentation end-products can be removed at intermediate points in the tube or at the top of the tube (the end remote from the feed end). The apparatus also allows for effluent (micro-organism) recycle and can include a separation device(s) in the recycle loop. The motor that causes the oscillation of liquid in the tube is preferably connected to the tube by a downwardly inclined pipe, to prevent settling of particles clogging the motor.

For facultative and obligately anaerobic microorganisms the major carbon source in the feed material can be, for instance, ground plant material such as straw, and/or soluble material such as molasses. This can be fermented to give microbial cells, extra-cellular enzymes and useful end-products such as hydrogen, methane, volatile fatty acids such as acetate, propionate and butyrate, succinate, ethanol, butenol and lactic acid.

For a better understanding of the invention, embodiments will now be described with reference to the attached drawing, in which:

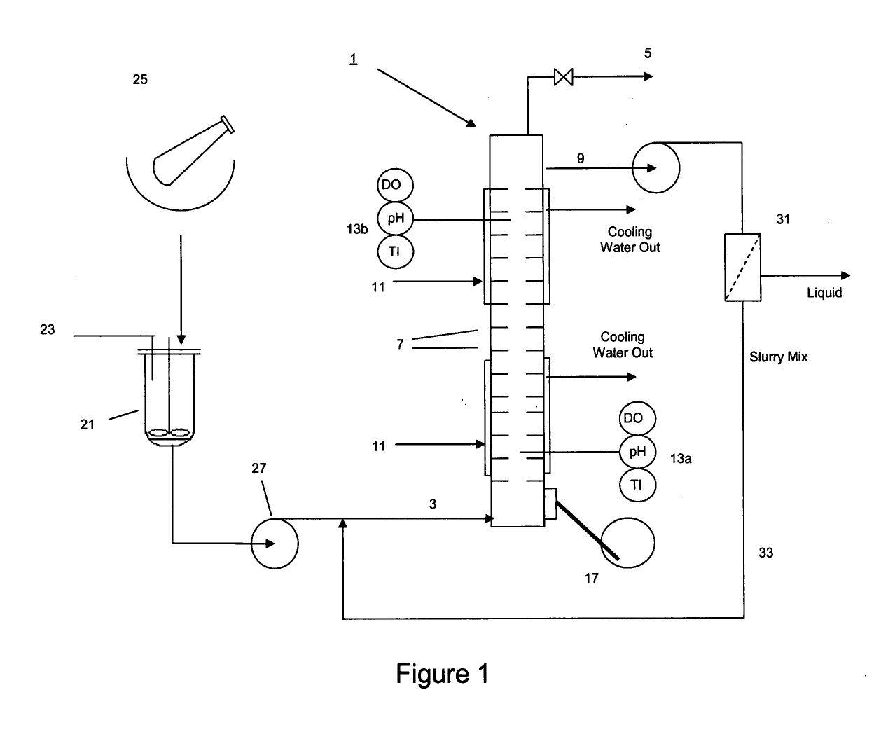

FIG. 1 shows apparatus embodying the invention;

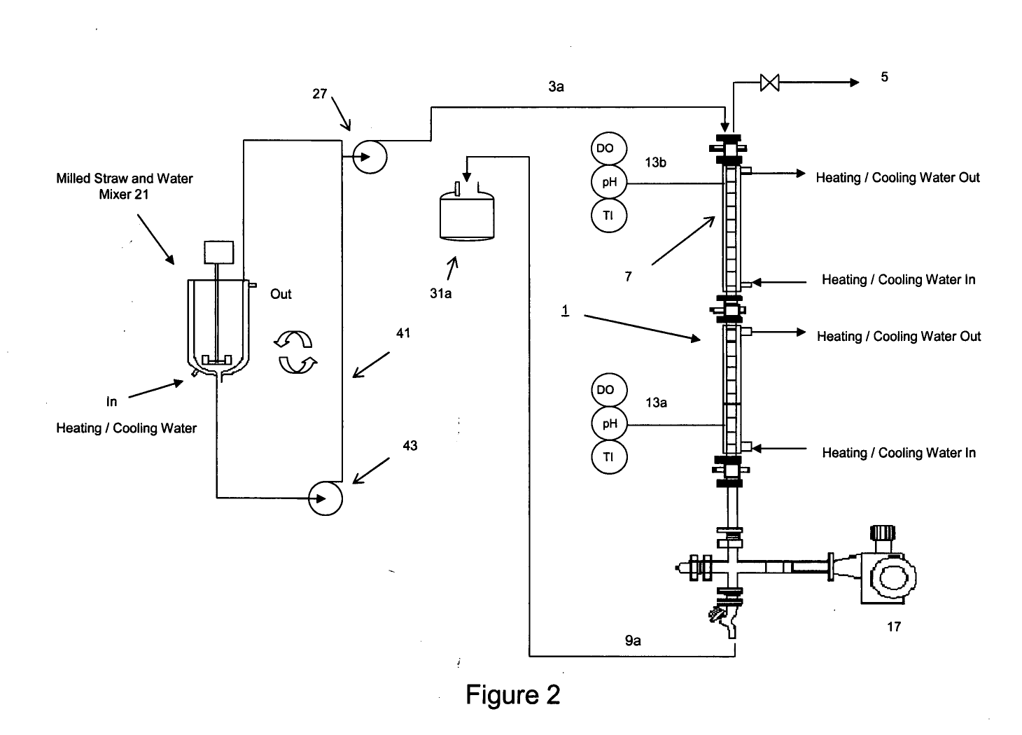

FIG. 2 shows a variant;

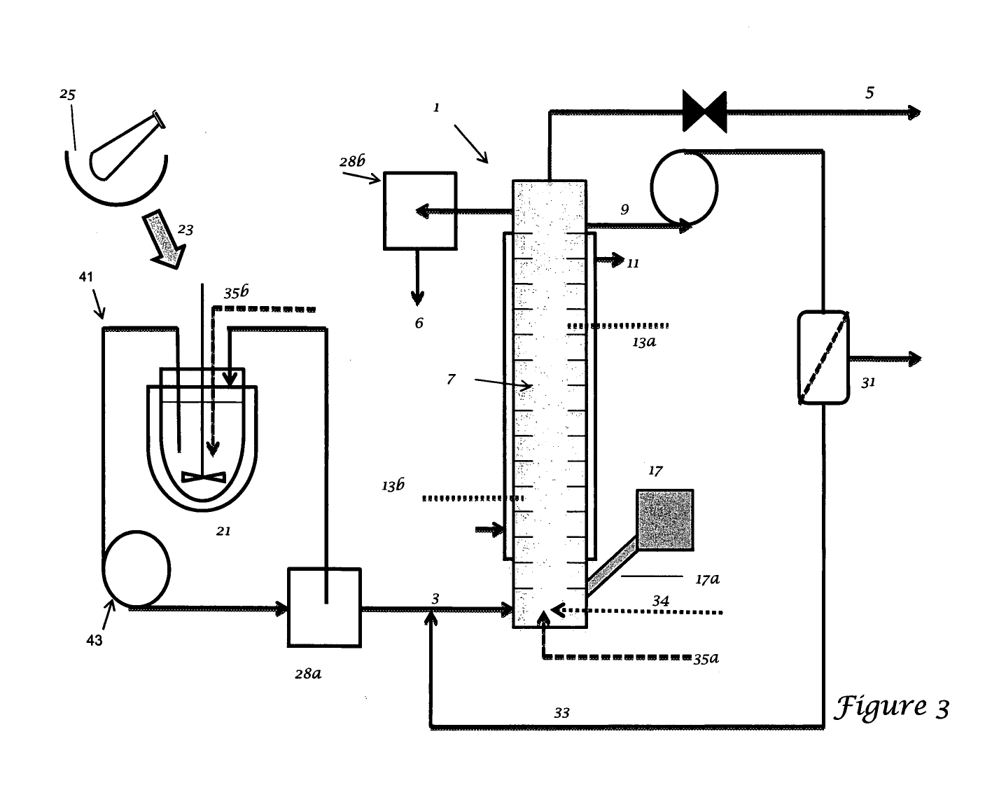

FIG. 3 shows a further development with further refinements;

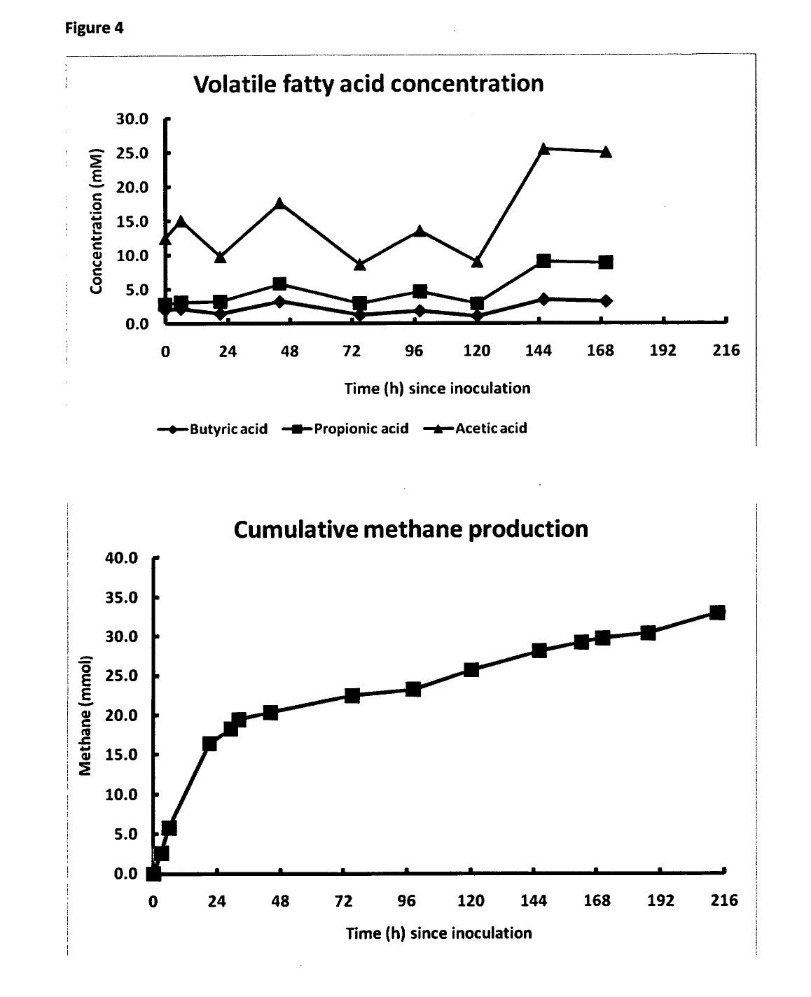

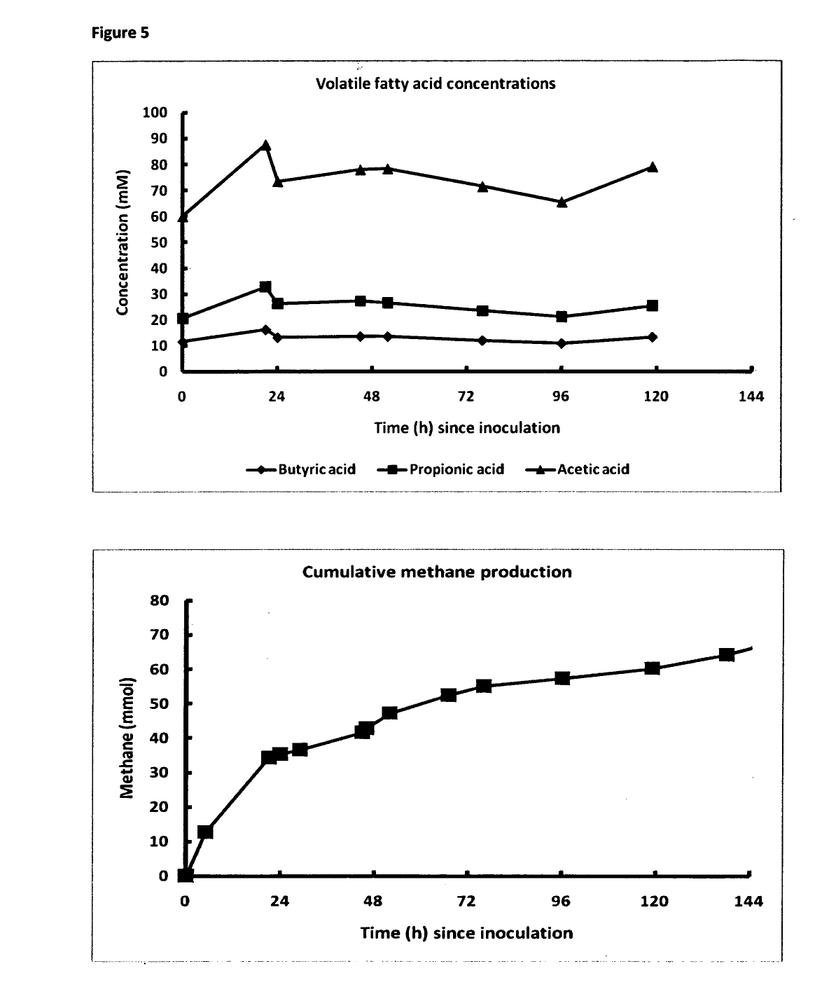

FIGS. 4 and 5 show experimental data; and

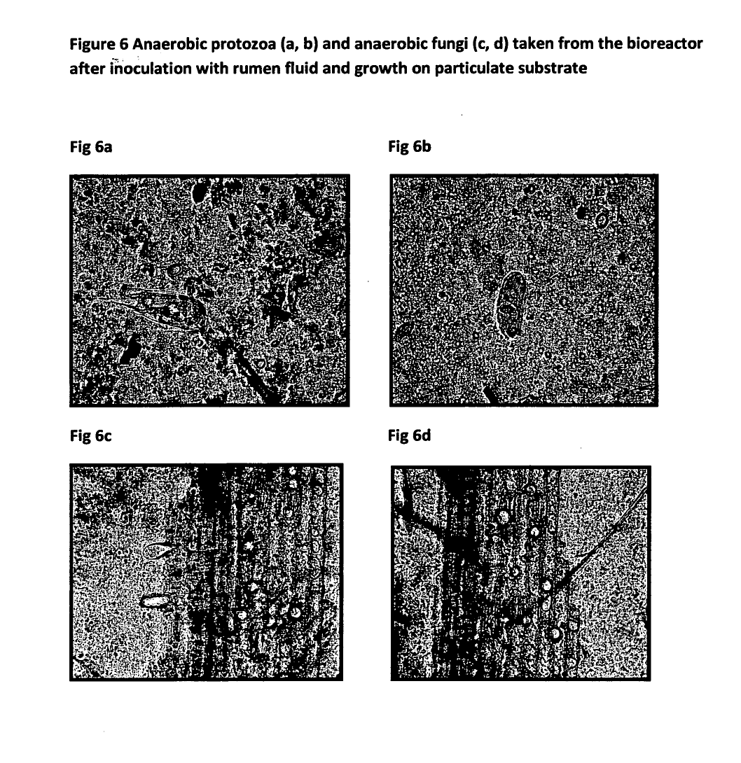

FIG. 6 shows photographs taken from the experiment.

### DETAILED DESCRIPTION

### Apparatus

FIG. 1 shows a vertical tube 1 having an inlet 3 at the bottom and outlets 5, 9 at the top as is known for continuous oscillatory flow baffle (COB) reactors. Over the length or height of the tube 1 and perpendicular to its axis annular baffles 7 are fixed at regular internals. The spacing is about half a baffle diameter along most of the length of the tube. The annular baffles have a central aperture of about a third of the diameter of the tube. Additional inlets can be made at all points along the tube to introduce other materials or gases to maintain the optimum environment required at any point in the process. Around the outside of the tube there are one or more constant-temperature jackets 11 through which water flows in order to control the temperature of the contents. Probes 13a, 13b are provided, here near the top and bottom, for such variables as temperature, pH and redox (Eh) potential, to be input into the control loop.

The supply of nutrient into the bottom of the reaction tube 1 comes from a vessel 21, which is fed with nutrient solution 23 inoculated with obligately or facultatively anaerobic microorganisms (e.g. from the rumen or silo) and particles from a comminuting apparatus 25. These particles can be plant material, for instance ground hay or straw or waste material. They are brought into the suspension by stirring in the container 21 and forwarded at intervals by a pump arrangement 27 and a valve (not shown) to the inlet 3.

Another pump 17 is connected to the base of the tube 1 and can be controlled so that the flow, although it is overall through the tube from the bottom to the top, also oscillates backwards and forwards. The liquid passes back and forth over the baffles, which generate vortices and thus ensure thorough mixing of contents and an even temperature distribution.

The gaseous end-products coming out of the reaction exit at the gas out port 5 at the top and can be collected for fuel cells, combustion or other purposes, while the liquid, containing microbial biomass, extra-cellular enzymes and soluble fermentation end-products, can be extracted via the outlet 9 into a vessel such as a separation device or filter 31. The filtered liquid or a part of it, or the solids/slurry, can be recycled as shown at 33, while soluble products can be extracted. This effluent recycling is useful to put back any particulate matter that has floated to the top, e.g. as a result of entrainment of gas bubbles.

The microorganisms are preferably cultures of rumen or silage organisms taken from the cow rumen or silage clamp respectively. The bacteria and fungi from these ecosystems produce microbial biomass, extra-cellular enzymes and valuable fermentation end-products as described above. The enzymes from some of these micro-organisms have the ability to degrade plant biomass to monomeric sugars which can be fermented to liquid fuel stocks such as ethanol. Such processes are restricted in conventional batch systems because microbial growth is limited by oxygen diffusion and by the build-up of product. The oscillating-baffle fermentor however can be controlled to ensure that conditions are suitable at all times. It is preferably operated continuously, although modified batch process can be used. It is also possible to connect the fermentor directly to feed hydrogen or methane fuel cells.

Other possible additives are agents to control the redox potential, themselves possibly bacteria such as Escherichia coli or Streptococcus bovis, or chemical reducing agents such as sodium sulphide or cysteine hydrochloride.

The baffle plates 7 would generally be smooth and resistant to colonisation. However, they could instead have a finely divided or porous surface to encourage growth, provided that excessive build-up of microbial biomass was prevented.

FIG. 2 shows an apparatus similar to that of FIG. 1 but with a few modifications. Similar or identical reference numerals are used as appropriate. On the feed side there is a recirculation loop 41 with its pump 43, for maintaining suspension of particles in the mixer 21 and therefore the feed. On the other hand there is no recirculation of product shown—the product is collected in a vessel 31a. Also, although the tube is still vertical, flow is from top to bottom, with feed 3a and an outlet 9a. Since the particulates have a tendency to settle, gravity assists the process.

FIG. 3 shows a third embodiment having a tube 1 (which is again vertical but could be horizontal) having an inlet 3, 6 once again at the bottom, and three outlets at 5 (for gas) (for effluent) and 9 (for recycle) at the top. Around the outside of the tube there is a constant-temperature jacket 11 through which water flows in order to control the temperature of the contents. Probes 13a, 13b are again provided for measurement and control of such variables as temperature, pH and redox (Eh) potential. This setup has both an input recycling loop 41 and an effluent recycling loop 33.

The supply into the bottom of the reaction tube 1 comes from a chilled medium reservoir 21, which contains a nutrient solution 23 prepared to include particles 25 (≦2 mm² and/or soluble (e.g. molasses) substrates. The particles can be plant material, for instance ground hay or straw, typically generated by milling and passage through a 1 or 2 mm dry mesh screen. They are brought into the suspension by a stirrer in the container 21 and circulated in a loop 41 by a fast circulating pump arrangement 27 having a flow rate of about 2.6 l min⁻¹. The medium supply is injected in precise quantities and at precise times to the reactor from the fast circulating loop through a valve arrangement 28a. This is linked to a second, ejection valve 28b which operates in synchrony with the injection valve 28a to ensure ejection of an equivalent quantity of spent material from the reactor. This material can be used as a nitrogen-rich fertilizer or for compost, for instance. The valves are preferably pneumatic valves, with a switching time of just a few ms and variable (ms-h) in switching time frequency. The use of these valves enables the system to be kept closed and hence anaerobic.

The oscillatory pump 17 is positioned appropriately to prevent clogging with particulate substrates. Here it is positioned above the base of the tube, to which it is connected by a descending pipe 17a. It can be controlled so that the flow, although it is generally through the tube from the bottom to the top, also oscillates backwards and forwards as before, keeping the particles in suspension.

Also at the base of the tube 1 are an inoculation point 34 for introducing organisms at the beginning of a run and from time to time, and a gas inlet 35a if required, e.g. for sparging.

The gaseous end-products coming out of the reaction exit at the gas out port 5, e.g. via a water trap, and can be collected for fuel cells, combustion or other purposes, while the liquid, containing microbial biomass, extra-cellular enzymes and soluble fermentation end-products, can be extracted via the effluent out port 6, being replaced by a fresh “plug” as described above.

The reactor also allows for effluent recycle through the recycle loop 33 which includes an outlet 9 and a separation device or filter 31. This is mainly to ensure that particles that float to the top can be returned to suspension, with the associated microorganisms. Not shown is a gas buffer or space at the top of the tube.

Although vertical tubes are preferred, as shown in the diagrams, horizontal or inclined tubes, or multiple serpentine tubes, are not ruled out.

### Microbes

The microorganisms are mixed communities or axenic cultures of obligate and/or facultative anaerobes generally involved in fermentation or anaerobic digestion processes. Some may require a phase of oxygenated growth in the reactor in order to produce biomass. For the purpose of demonstrating application of the reactor, data will be demonstrated from the growth of a consortium of obligately anaerobic microorganisms taken from the rumen digesta of a non-lactating dairy cow. The obligately anaerobic bacteria, protozoa and fungi from this ecosystem are extremely effective in degrading recalcitrant lignocellulosic substrates to produce microbial biomass, extra-cellular enzymes and valuable fermentation end-products as described above. Indeed, the enzyme system from the anaerobic fungal genus Neocallimastix in this community is reported to be the most active known to man. The fungi, collectively with other fibrolytic members of the consortium, degrade plant biomass to its constituent monomers and ferment these to primary end-products, some of which can then serve as substrates for the rumen (and other) methanogens for the production of methane. Other processes appropriate for the reactor include the production of liquid fuel stocks such as ethanol from yeast.

### Experimental Data

The reactor configuration used to generate the experimental data provided in support of this application is the variant shown in FIG. 3 and the data itself are presented in FIGS. 4-6. Two experiments are presented and the conditions used to generate the data shown in FIG. 4 for the first experiment were as follows:

The operating volume (liquid volume) and temperature of the reactor was 800 ml and 39° C. respectively. The pH was controlled at 6.85±0.1 and the redox potential (Eh), although not recorded for this experiment, was between 150 and 300 mV as imparted by the rumen fluid inocula on start-up and the reducing agent used in the ‘artificial saliva’ culture medium.

The piston of the oscillatory baffle apparatus was operated at a frequency of 110 Hz in order to keep plant biomass particles in suspension and well mixed throughout the reactor. At this frequency, and in the vertical position, the reactor was not operating under plug flow conditions.

The inoculum used at start-up consisted of 200 ml of fresh rumen fluid collected from a hay-fed cow. The rumen fluid was strained through 4 layers of muslin and mixed with 8 g (fresh weight) of solid digesta contents. The inoculum was added to the reactor which contained 600 ml artificial saliva and 4 g barley straw and 4 g hay (both milled to pass through a 2 mm dia dry mesh screen). Both the straw and the hay had a dry matter content of approximately 90-95%. Continuous sparging with CO₂, shown at 35b, ensured the maintenance of anaerobic conditions during addition to the reactor.

The artificial saliva was from a standard recipe often used in rumen microbiology/ruminant nutrition and was of the following composition (gl⁻¹): Na₂HPO₄ 3.6; NaHCO₃ 9.6; NaCl 0.46; KCl 0.56; CaCl₂ 0.04; MgCl₂ 0.05. The reactor was fed from five litres of additional artificial saliva that contained (NH₄)₂SO₄ 0.9 gl⁻¹ in addition to the above chemicals and was mixed with particles of barley straw (10 gl⁻¹) and hay (10 gl⁻¹) milled as above and held at 4-6° C. in the medium reservoir and fast circulation loop. Sparging with N₂, shown at 35a, and the appropriate use of reducing agents ensured the maintenance of anaerobic conditions in the equipment. In some experiments the redox (Eh) indicator, resasurin, was used to show that the artificial saliva in the medium reservoir and reactor contents were sufficiently reduced to permit the growth or obligately anaerobic rumen microorganisms.

The data shown in FIG. 4 were obtained by operating the reactor in batch mode for 44 h, pH control by PID (proportional-integral-derivative) using artificial saliva according to the following interval: initial 6 h no control, followed by 15 h pH control by PID (125 ml artificial saliva added), 11 h of no control and a following 12 h pH control by PID (150 ml artificial saliva added). Following batch operation with pH control, the reactor was operated using semi-continuous pulse feeding, averaging 1 pulse of 88 ml of feed at 24 h intervals. The pH was controlled manually as appropriate during the pulse feeding phase of the reactor.

The results presented in FIG. 4 show volatile fatty acid concentrations and cumulative methane production in samples taken from the reactor over a 7-9 day period (and beyond but the data are not shown). The volatile fatty acids, acetate, propionate and butyrate are typical of those produced in the rumen and methane is also a typical end product of rumen fermentation. The molar proportions, acetate: propionate: butyrate are within the range expected for rumen fermentations, although in this particular experiment the amounts produced are lower than expected. However, by adjusting reactor conditions, particularly the feeding regime, it was possible to produce data with significantly elevated levels of volatile fatty acids and methane.

The data shown in FIG. 5, for example, demonstrate that it is possible to obtain volatile fatty acid concentrations in the reactor that are approximately the same as those produced in the rumen. While methane is approximately 2-fold higher, the concentration of volatile fatty acids shown in FIG. 5 is approximately five-fold higher than those recorded in the experiment depicted in FIG. 4. The fact that volatile fatty acids and methane are being generated by the reactor in a reasonably constant fashion and from fibrous substrates is indicative of the presence of a consortium of fibrolytic rumen microorganisms, which includes Eubacteria and Archaea mentioned above. Light microscopy of reactor contents also confirmed the presence of Eukaryotes, anaerobic fungi and protozoa in the reactor (FIG. 6). For the anaerobic fungi, the presence of sporangia on straw and hay particles and motile zoospores in the liquid phase indicated that they were viable and able to grow in the reactor.

In conclusion, these results show that we have invented a reactor and process methodology which enables us to mimic the rumen ecosystem and to grow a resilient consortium of obligately anaerobic micro-organisms continuously and outside of their natural ecosystem. From the production rate of methane and volatile fatty acids seen in these experiments, the rate of degradation of particulate matter in the column is likely to be similar to that recorded in numerous short-term batch culture studies that employ mixed rumen inocula (see for example, Theodorou et. al., 1994 Animal Feed Science and Technology, 48 185-197 and references therein). The rate and extent of degradation in the reactor will be well in excess of that achieved in a conventional (clostridium-based) anaerobic digestion process.

The reactor has considerable potential and versatility and can be used for a range of microbiological applications. The continuous or semi-continuous mode of operation, the method of feeding by injecting and ejecting particles via the fast loop/synchronous valve configuration, the ability to keep particles in suspension by using oscillatory baffles and the small size of the reactor in comparison to stirred tank fermenters and anaerobic digesters, provide considerable advantages in microbiological application and process design.

1. A method for controlling an anaerobic fermentation process, comprising the steps of:
providing an oscillatory flow reactor (1) having a reaction tube with baffles (7) along its length;
introducing (3) fluid material into the tube under anaerobic conditions, the fluid comprising the following components:
a finely divided substrate material;
facultative and/or obligate anaerobic organisms, and
feed material for the organisms, the feed possibly being wholly or partly identical to the substrate;
passing the fluid material through the tube at a speed that allows anaerobic growth on and fermentation of the feed material to take place, maintaining the anaerobic conditions while causing or allowing the fluid to oscillate with respect to the baffles; and
collecting the fermentation products.

2. The method according to claim 1, in which the finely divided substrate material includes particulate material in suspension.

3. The method according to claim 2, in which the particulate substrate material includes comminuted plant material.

4. The method according to claim 1 and being a continuous process.

5. The method according to claim 1, in which the products extracted include fuels, microbial biomass, platform chemicals or extra-cellular enzymes.

6. The method according to claim 1, in which the organisms include rumen or silage organisms.

7. The method according to claim 1, in which the finely divided material is kept in suspension in a reservoir (21) and injected intermittently into the reactor.

8. An oscillatory flow reactor, comprising:
a tube (1) with feed (3) at one end and discharge (5, 6, 9) at the other, and a baffle-plate system (7) arranged along its length for mixing the contents of the tube by oscillation of the contents with respect to the plates;
further including a supply system (21, 27, 34, 41) for feeding to the tube a suspension of substrate particles, facultative and/or obligate anaerobic organisms and feed material under anaerobic conditions, the supply system including a valve system for keeping the tube closed and hence anaerobic during the feed and the reaction processes.

9. An oscillatory flow reactor, comprising:
a tube (1) with feed (3) at one end and discharge (5, 9) at the other, and a baffle-plate system (7) arranged along its length for mixing the contents of the tube by oscillation of the contents with respect to the plates;
further including a supply system (21, 27) for feeding to the tube facultative and/or obligate anaerobic organisms and feed material under anaerobic conditions, and the baffle plates (7) have a finely divided surface on which the organisms can grow and including a valve system for keeping the tube closed and hence anaerobic during the feed and the reaction processes.

10. The reactor according to claim 8, further including a temperature-control device (11) and/or a redox potential probe (13), for use in controlling the reaction conditions.

11. The reactor according to claim 8, including a reservoir (21) for the particle suspension, a suspension loop (41) and a pump (27; 43) for the feed flow in the loop to keep the solids in suspension, and a valve system (28a) in the loop to allow intermittent injection into the tube.

12. The reactor according to claim 11, further including an effluent outlet (6) opened by a valve system (28b) synchronized with the particle-injection valve system (28a).

13. The reactor according to claim 8, including a pump (17) for the oscillation of the flow in the tube.

14. The reactor according to claim 8, further including a recycling loop (9, 31, 33) for returning microorganisms and other contents from the outlet end of the tube to the inlet end.

15. The reactor according to claim 8, in which the tube is vertical.
